(19) United States
(12) Patent Application Publication
(10) Pub. No.: US 2026/0345085 A1
(43) Pub. Date: Nov. 5, 2026

(54) A COMPUTER-IMPLEMENTED METHOD, DEVICE, COMPUTER PROGRAM PRODUCT AND COMPUTER-READABLE STORAGE MEDIUM FOR IDENTIFYING PRESSURE ULCERS ON TISSUE OF A SUBJECT

(71) Applicant: TECHNISCHE UNIVERSITEIT EINDHOVEN, EINDHOVEN (NL)

(72) Inventor: Geert Hein VAN KOLLENBURG, EINDHOVEN (NL)

(21) Appl. No.: 19/140,062

(22) PCT Filed: Dec. 18, 2023
(86) PCT No.: PCT/NL2023/050667

§ 371 Date: Jun. 17, 2025

(30) Foreign Application Priority Data
Dec. 22, 2022 (NL) 2033799

(51) Int. Cl. A61B 5/00

(52) U.S. Cl. CPC A61B 5/7267; A61B 5/0075; A61B 5/445; A61B 2560/0431

(57) ABSTRACT

The above mentioned and other objects are achieved, in the first aspect of the present disclosure, by a computer-implemented method for identifying pressure ulcers on tissue of a subject with a handheld scanner, the method comprising the steps of: obtaining sample data from the handheld scanner when placed against the tissue of the subject, wherein the sample data comprises radiation intensity values of radiation emitted by the handheld scanner which reflected of the tissue; profiling the sample data, thereby obtaining a reflection spectrum of the sample; classifying the reflection spectrum using a machine-learning (ML)-classifier thereby obtaining a classification score for identifying the pressure ulcers at the tissue of the subject; presenting the classification score by the handheld scanner; wherein the ML-classifier is trained with a training set, wherein the training is performed in a training routine; and wherein the training routine comprises training the ML-classifier with a Random Subspace Ensemble routine.

### TECHNICAL FIELD

The present disclosure relates to a computer-implemented method and a device for identifying pressure ulcers on tissue of a subject.

The present invention further relates to a computer program product and computer-readable storage medium for performing the method for identifying pressure ulcers on tissue of a subject.

### BACKGROUND

Pressure ulcers or sores are areas of damaged skin and tissue that can develop when a person remains in the same position for too long. This can happen when a person is bedridden or otherwise unable to move freely, and is most common among older adults, people with chronic illnesses, and those with mobility impairments. Sores can develop through pressure, friction/shear and also moisture (or combinations of these), all of which make that blood supply is cut-off to that tissue.

There are several devices available for detecting pressure ulcers, including pressure mapping systems, which use sensors to measure the pressure being applied to different parts of the body, and shear force sensors, which measure the forces that can cause skin damage.

A disadvantage of these devices is that they can be expensive, complex and may not be practical for widespread use. Moreover, such devices may not be accurate in detecting (early-stage) pressure ulcers, which can make it difficult to intervene before the ulcers become more severe.

Another disadvantage of these known devices is that the use thereof is experienced as very unpleasant for the subject, for example typically someone who is being cared for in a nursing home, or someone who has been in a hospital bed for a long time. Known devices, such as the device for diagnosing pressure ulcers disclosed in U.S. Pat. No. 8,116,838 B2, are based on applying pressure to the tissue, which results in a change in the color of the tissue under the influence of this pressure. On the basis of this change, the device is able to determine whether there are pre-existing pressure ulcers or pressure ulcers that are already at an advancing or advanced stage. Early detection is challenging with such devices because the tissue still shows little or no external characteristics after pressure is exerted on the tissue, from which it can be determined with a high degree of certainty that there are pressure ulcers. Because ulcers are very sensitive, even at an early stage, let alone exerting significant pressure on them for testing, this detection technique is experienced as very unpleasant. Known devices are also typically complex and therefore expensive, which might require training before usage and make the use cumbersome.

There is therefore a need to provide an improved detection method in which one or more of the aforementioned problems are resolved.

### SUMMARY

The above mentioned and other objects are achieved, in the first aspect of the present disclosure, by a computer-implemented method for identifying pressure ulcers on tissue of a subject with a handheld scanner, the method comprising the steps of:
a) obtaining sample data from the handheld scanner when placed against the tissue of the subject, wherein the sample data comprises radiation intensity values of radiation emitted by the handheld scanner which reflected of the tissue;
b) profiling the sample data, thereby obtaining a reflection spectrum of the sample;
c) classifying the reflection spectrum using a machine-learning (ML)-classifier thereby obtaining a classification score for identifying the pressure ulcers at the tissue of the subject;
d) presenting the classification score by the handheld scanner;
wherein the ML-classifier is trained with a training set, wherein the training is performed in a training routine; and
wherein the training routine comprises training the ML-classifier with a Random Subspace Ensemble routine.

The training routine or classifier is in particular a ML-classifier based on or comprising a Random Subspace Ensemble routine. A random subspace ensemble is a machine learning technique that involves training multiple models on different random subsets of the features in a dataset, and then combining the predictions of those models to make a final prediction. This approach can be used to improve the performance of a model by reducing overfitting and increasing the diversity of the models being trained.

One advantage of using a random subspace ensemble is that it can help to improve the performance of a model by increasing the diversity of the models being trained. This can be particularly useful when dealing with complex, high-dimensional datasets where overfitting is a concern. Additionally, because random subspace ensembles involve training multiple models on different subsets of the features in a dataset, they can be computationally efficient and can be implemented using a distributed computing approach, which can further improve their performance.

On the other hand, one disadvantage of using a random subspace ensemble is that it can be more difficult to interpret and explain the predictions made by the resulting model compared to a single model trained on the entire dataset. Additionally, because random subspace ensembles involve training multiple models, they can be more computationally expensive than a single model, which can be a disadvantage in situations where computational resources are limited.

The present disclosure is based on the insight that such ML routines, and in particular Random Subspace Ensemble routines or the like, benefit from high scoring accuracy for the classification score, but on the other hand, require, in comparison with other known ML routines, relatively limited processing resources, which allows the routine to be build-in into a handheld scanner which is portable and increases the ease in use, without requiring the tissue of the subject to be subject to pressure, which is a required step in the techniques currently known. As such, the proposed method gives less discomfort, is considered more pleasant, and less cumbersome in use.

In a second aspect of the present disclosure a handheld device is provided for identifying pressure ulcers on tissue of a subject with a handheld scanner, the device comprising a radiation source, a radiation detector, indication means and a processing unit, wherein the radiation source and radiation detector are arranged to be placed against the tissue of the subject, for the radiating detector to obtain sample data, wherein the sample data comprises radiation intensity values of radiation emitted by the handheld scanner which reflected of the tissue, and wherein the processing unit is arranged for profiling the sample data, thereby obtaining a reflection spectrum of the sample, and classifying the reflection spectrum using a machine-learning (ML)-classifier thereby obtaining a classification score for identifying the pressure ulcers at the tissue of the subject, and presenting, with the indication means, the classification score to a user of the device, and wherein the ML-classifier is trained with a training set, wherein the training is performed in a training routine; and the training routine comprises training the ML-classifier with a Random Subspace Ensemble routine.

In a third aspect of the present disclosure a computer program product is provided, comprising instructions which, when the program is executed by a computer, cause the computer to carry out the steps of the method of any preceding descriptions. In a fourth aspect of the present disclosure a computer-readable storage medium, comprising instructions which, when executed by a computer, cause the computer to carry out the steps of the steps of the method of any preceding claims descriptions.

In a further aspect, there is disclosed a computer-implemented method for identifying features in an image of a tissue of a subject, wherein said image is generated by a handheld scanner, the method being performed by the handheld scanner and comprising the steps of: a) obtaining sample data from the handheld scanner when placed against the tissue of the subject, wherein the sample data comprises radiation intensity values of radiation emitted by the handheld scanner which reflected of the tissue;
b) profiling the sample data, thereby obtaining a reflection spectrum of the sample;
c) classifying the reflection spectrum using a machine-learning (ML)-classifier thereby obtaining a classification score for identifying features at the tissue of the subject;
d) presenting the classification score by the handheld scanner;
   wherein the ML-classifier is trained with a training set, wherein the training is performed in a training routine; and wherein the training routine comprises training the ML-classifier with a Random Subspace Ensemble routine.

The ML classifier, in particular the Random Subspace Ensemble, can be applied to classify a tissue of a subject. In general, the method according to the present disclosure may be suitable for a wide range of applications to identify tissue and classify healthy tissue from tissue specific abnormalities and diseases. In particular, the method is arranged and configured for one or more of ulcers, bedsores, decubitus, but also osteomyelitis, inflammation (with or without infection), or other tissue identifiable abnormalities and diseases. To this end, in the final step of the method, a classification score is presented which represents the likelihood that the abnormality or disease is present, and preferably, at which stage of development the abnormality or disease is classified.

The computer-implemented method may be configured for ulcers, by training the ML classifier accordingly with such a ulcer based data set. However, the method may also be configured for other applications with respective data sets such as osteomyelitis, inflammation, or other tissue identifiable abnormalities and diseases

In the context of the present disclosure, deep learning and particularly convolutional neural networks (CNNs), have been found to be well-suited for such specific tissue analysis. To recognize elements on the skin or in the tissue, such as abnormalities or disease indicators, relevant ‘features’ are employed. These features may encompass color variations, texture, lesion size, and other morphological properties. In skin analysis, the Random Subspace Ensemble routine has been found to be very effective to identify and analyze such features, define learning patterns and diagnose specific tissue conditions.

The use of the Random Subspace Ensemble routine in the proposed computer-implemented method for identifying conditions, abnormalities and diseases such as, but not limited to pressure ulcers, on tissue of a subject through a handheld scanner represents a surprisingly highly effective choice, particularly when considering its application to various conditions. Exemplary embodiments of the present disclosure benefit from the surprising effectiveness of this technique, especially for small handheld devices. Unlike traditional methods that may be computationally expensive or challenging to interpret, the Random Subspace Ensemble routine has some unique advantages. Ensemble learning is very powerful yet on the one hand surprisingly well-suited for nuanced and complex nature of such tissue conditions, and on the other hand, fast, reliable but also aligns with the resource constraints inherent in portable, handheld devices. With the proposed routine, the challenges associated with overfitting in high-dimensional datasets can be addressed. The Random Subspace Ensemble routine adaptability extends beyond the application of pressure ulcers, making it a versatile and valuable tool for identifying various tissue-specific abnormalities and diseases, such as osteomyelitis, inflammation, or other tissue or skin-related conditions.

The method and corresponding device, more in particular, handheld scanner, preferably employes near-infrared light to detect the unique chemical composition of the tissue. To this end, the scanner placed against the tissue and the light from the scanner, when reflected from the tissue, is collected and analyzed accordingly by the ML classifier. After the analysis is then completed, an indication is given of the likelihood that the condition is present. Any example or embodiment of the first aspect of the present disclosure is also applicable and suitable for the above further aspect, and for the second, third and any other aspects of the present disclosure.

In any of the above mentioned aspects of the present disclosure a feature is defined wherein the sample data is obtained from the handheld scanner when placed against the tissue of the subject, wherein the sample data comprises radiation intensity values of radiation emitted by the handheld scanner which reflected of the tissue. For each of these aspects, or preferably at least in the aspects of the present disclosure that relate to a method, the sample data may alternatively be received, thereby indicating that there is no interaction with the actual tissue. In such an example, the data creation is outside of the scope of the claimed invention, whereas the data, once generated, is received by and analyzed only.

For each of the aspects of the present disclosure that relate to a method, the method steps are in an example, performed in a physical entity, preferably in the handheld device.

In an example of any of the aspects of the present disclosure, the handheld scanner is a Near-InfraRed, NIR, scanner for emitting light in a wavelength range within the near-infrared spectrum, and in particular in the wavelength range of approximately 800 to 2500 nm, more in particular in the range of approximately 900 1700 nm, and most in particular in the range of approximately 950-1650 nm. The NIR region is from around 800 to 2500 nm. Higher regions may be used, but are more difficult (i.e., more costly) to implement in a handheld sensor.

In an example of any of the aspects of the present disclosure, the handheld scanner is operated in a diffuse reflectance mode to obtain sample data from which comprises radiation intensity values of radiation emitted by the handheld scanner with an evenly distributed spectral resolution, more in particular at 125 variables per measurement.

In an example of any of the aspects of the present disclosure, the ML-classifier with Random Subspace Ensemble routine is trained.

In an example of any of the aspects of the present disclosure, the training routine is generated in accordance with a duplex method.

In an example of any of the aspects of the present disclosure, all the sample data obtained in step a) is assigned to either a training set or a test set, and wherein the training set comprises approximately 70% of the sample data and the test test approximately 30% of the sample data.

In an example of any of the aspects of the present disclosure, training routine comprises training the ML-classifier with a Random Subspace Ensemble routine in which the reflection profile is divided into a discrete number of random subspaces.

In an example of any of the aspects of the present disclosure, the discrete number of random subspaces is approximately at least 10, more preferably approximately at least 20, most preferably approximately at least 30.

In an example of any of the aspects of the present disclosure, the ML-classifier comprises a selection of, or all of the subspaces are combined in a combined classifier.

In an example of any of the aspects of the present disclosure, the ML-classifier is trained with the training routine set with an epoch of at least 50, preferably at least 75, more preferably at least 100 epochs, most preferably at least 150 epochs.

In an example of any of the aspects of the present disclosure, the ML-classifier comprises approximately at least 10 neurons, preferably at least 20 neurons, more preferably at least 30 neurons, and most preferably at least 40 neurons.

The present disclosure is described in conjunction with the appended figures. It is emphasized that, in accordance with the standard practice in the industry, various features are not drawn to scale. In fact, the dimensions of the various features may be arbitrarily increased or reduced for clarity of discussion.

In the appended figures, similar components and/or features may have the same reference label. Further, various components of the same type may be distinguished by following the reference label by a dash and a second label that distinguishes among the similar components. If only the first reference label is used in the specification, the description is applicable to any one of the similar components having the same first reference label irrespective of the second reference label.

The above and other aspects of the disclosure will be apparent from and elucidated with reference to the examples described hereinafter.

### BRIEF DESCRIPTION OF THE DRAWINGS

FIG. 1 shows the steps of the method according to a first aspect of the present disclosure;

FIG. 2 shows a device according to a second aspect of the present disclosure.

### DETAILED DESCRIPTION

It is noted that in the description of the figures, same reference numerals refer to the same or similar components performing a same or essentially similar function.

A more detailed description is made with reference to particular examples, some of which are illustrated in the appended drawings, such that the manner in which the features of the present disclosure may be understood in more detail. It is noted that the drawings only illustrate typical examples and are therefore not to be considered to limit the scope of the subject matter of the claims. The drawings are incorporated for facilitating an understanding of the disclosure and are thus not necessarily drawn to scale. Advantages of the subject matter as claimed will become apparent to those skilled in the art upon reading the description in conjunction with the accompanying drawings.

The ensuing description above provides preferred exemplary embodiment(s) only, and is not intended to limit the scope, applicability or configuration of the disclosure. Rather, the ensuing description of the preferred exemplary embodiment(s) will provide those skilled in the art with an enabling description for implementing a preferred exemplary embodiment of the disclosure, it being understood that various changes may be made in the function and arrangement of elements, including combinations of features from different embodiments, without departing from the scope of the disclosure.

Unless the context clearly requires otherwise, throughout the description and the claims, the words “comprise,” “comprising,” and the like are to be construed in an inclusive sense, as opposed to an exclusive or exhaustive sense; that is to say, in the sense of “including, but not limited to.” As used herein, the terms “connected,” “coupled,” or any variant thereof means any connection or coupling, either direct or indirect, between two or more elements; the coupling or connection between the elements can be physical, logical, electromagnetic, or a combination thereof. Additionally, the words “herein,” “above,” “below,” and words of similar import, when used in this application, refer to this application as a whole and not to any particular portions of this application. Where the context permits, words in the Detailed Description using the singular or plural number may also include the plural or singular number respectively. The word “or,” in reference to a list of two or more items, covers all of the following interpretations of the word: any of the items in the list, all of the items in the list, and any combination of the items in the list.

These and other changes can be made to the technology in light of the following detailed description. While the description describes certain examples of the technology, and describes the best mode contemplated, no matter how detailed the description appears, the technology can be practiced in many ways. Details of the system may vary considerably in its specific implementation, while still being encompassed by the technology disclosed herein. As noted above, particular terminology used when describing certain features or aspects of the technology should not be taken to imply that the terminology is being redefined herein to be restricted to any specific characteristics, features, or aspects of the technology with which that terminology is associated. In general, the terms used in the following claims should not be construed to limit the technology to the specific examples disclosed in the specification, unless the Detailed Description section explicitly defines such terms. Accordingly, the actual scope of the technology encompasses not only the disclosed examples, but also all equivalent ways of practicing or implementing the technology under the claims.

Pressure ulcers (‘bedsores’, decubitus) can lead to tissue loss and life-threatening complications like blood poisoning if left untreated. Rapid identification of pressure ulcers is critical, but current diagnosis is done visually, which is difficult even for experienced healthcare professionals, especially in patients with darker skin tones. A (chemical) sensor that can identify subcutaneous ulcers based on chemical fingerprints of changing tissue can significantly improve early detection of ulcers and can be used to preventatively check parts of the body at risk of developing ulcers. Treatment of ulcers can also be a long process, resulting in high costs and severe discomfort in the patient.

In the US alone, about 2.5 million patients per year are affected by pressure ulcers, costing an estimated $11 billion per year (Sen et al 2009. Wound Repair Regen. 17, 763-771 (2009)). In hospitals if decubitus develops, the patient cannot be discharged until the decubitus has been healed, leading to long-term bed occupation with significant costs.

Many handheld sensors exist for various applications, but none are directly suited to identify ulcers. Academic studies on the detection of ulcers include:
‘smart bandages’ that continuously map pressure-induced tissue damage (based on impedance spectroscopy).
Spectroscopy has been evaluated, but had limited success in an exploratory study (investigating low-cost optical spectroscopy for sensing pressure ulcers—Smruti Suresh Mirchandani, miami University).
Several applications study moisture content in skin:
Adipose tissue can be identified:

### Optical Properties of the Subcutaneous Adipose Tissue

Near-infrared light is invisible to the human eye but the different ‘colors’ in this region of light are related to different chemical bonds. The proposed method and device, more in particular, scanner, preferably uses near-infrared light to detect the unique chemical composition of tissue related to bedsores/pressure ulcers. The scanner is gently held against a certain part of the body. The light from the scanner reaches the tissue where the ulcer may be developing. The reflected light is collected and can be directly analyzed. The scanner then indicates how likely it is that an ulcer is present.

The proposed method is arranged to be implemented into a hand held device, and more in particular, a device which can be used single-handed. The method is based on sampling a tissue of a subject, which may be a mammal and more in particular a human. With the sampling, sample data is obtained for further processing by the method or when implemented, the device. The sample data is obtained from a radiation source which emits radiation, preferably in the form of light, and more preferably, invisible light, even more preferably near-infrared light. A radiation detector, e.g. a photodetector or photodetector array in case of light or near-infrared light, is used to detect and sample the radiation received as reflection from the tissue of the subject which is under test.

A processing unit thereby obtains sample data containing radiation intensity values, which are used to generate a profile or in other words, profile the sample data. The sample data comprises a reflection spectrum of the sample. The reflection spectrum may then be pre-processed before the actual use by the classification routine. And the skilled person will appreciate which pre-processing routines may be applicable.

Now the reflection spectrum is ready for use by a classifier to obtain a classification score. The classifier is a machine learning classifier by which the classification score is obtained as indication for the presence of the pressure ulcers at the tissue under test.

The classification score is then presented by the handheld scanner for the user of the scanner to determine, in an easy manner, the indication whether or not the tested tissue shows signs of pressure ulcers. The way of presenting the score may be done in different ways, for example in a digital manner in which the actual score is presented, or in the form of lights having different indications or colors to indicate the classification score, and so on. The skilled person will appreciate which techniques may be suitable and applicable, which details go beyond the scope of the present disclosure.

The training routine or classifier is in particular a ML-classifier based on or comprising a Random Subspace Ensemble routine. A random subspace ensemble is a machine learning technique that involves training multiple models on different random subsets of the features in a dataset, and then combining the predictions of those models to make a final prediction. This approach can be used to improve the performance of a model by reducing overfitting and increasing the diversity of the models being trained.

One advantage of using a random subspace ensemble is that it can help to improve the performance of a model by increasing the diversity of the models being trained. This can be particularly useful when dealing with complex, high-dimensional datasets where overfitting is a concern. Additionally, because random subspace ensembles involve training multiple models on different subsets of the features in a dataset, they can be computationally efficient and can be implemented using a distributed computing approach, which can further improve their performance.

On the other hand, one disadvantage of using a random subspace ensemble is that it can be more difficult to interpret and explain the predictions made by the resulting model compared to a single model trained on the entire dataset. Additionally, because random subspace ensembles involve training multiple models, they can be more computationally expensive than a single model, which can be a disadvantage in situations where computational resources are limited.

The present disclosure is based on the insight that such ML routines, and in particular Random Subspace Ensemble routines or the like, benefit from high scoring accuracy for the classification score, but on the other hand, require, in comparison with other known ML routines, relatively limited processing resources, which allows the routine to be build-in into a handheld scanner which is portable and increases the ease in use, without requiring the tissue of the subject to be subject to pressure, which is a required step in the techniques currently known. As such, the proposed method gives less discomfort, is considered more pleasant, and less cumbersome in use.

The invention may be embodied by connecting a NIR sensor (e.g., MantiSpectra OEM Sensing Board) and a (miniature) OLED display to a control unit or processing unit, such as an Arduino board with LED light source. The NIR sensor and LED could together be fitted with a short tube of soft thermoplastic elastomer, with a stiffener to ensure no light comes directly from the LED to the NIR sensor. A software interface can turn on the LED when a scan is requested e.g. when a button is pressed, and then collect measurements from the NIR sensor. A calculation programmed into the device (based on previously obtained data analysis) then transforms the NIR data into a likelihood/probability of ulcer in the scanned area. For a prototype, a 3d-printed solution could be made to hold the components together.

The invention is however not limited to the above mentioned example of the NIR sensor, but may also be embodied by the use of more advanced sensor (like Spectral Engines' NIRONE® Sensor S, instead of the MantiSpectra, or through a Raman spectroscopy sensor) The skilled person will appreciate which sensors and measuring techniques may be suitable for obtaining a profile suitable for the ML-method as proposed by the present disclosure.

Because of the dedicated purpose of this sensor, an Arduino board has significant overcapacity. Simpler/custom alternatives could be used to lower production costs.

The Machine Learning, ML, method used is known as random subspace discriminant ensemble (RSDE). RSDE is implemented by splitting the spectra into multiple (random) smaller subsets. Each subset is then classified (i.e. decubitus present yes/no) separately with a machine learning model that performs best in that subset (e.g. random forest). The final classification is then done based on ‘majority vote’.

The proposed model or algorithm is beneficial for this particular application because the required computational complexity is significantly simplified, because the classification problem is split into multiple smaller parts. This is beneficial for resource-limited devices like a portable handheld sensing device.

The proposed sensor does not require a (live) connection with an external server(s) or other devices. Although such connection with a back-end server may in an example be implemented, it is thus not required and in basic form, the scanner is portable and handheld and stand-alone.

The software may be configured specifically for detecting ulcers and the sensor may have a display that informs the user directly about the results, or may have any other type of indication means. The part of the sensor that touches the body (i.e., the sample) is preferably made of soft non-toxic, hypoallergenic material, such as silicone.

Preferably, the device requires that a NIR sensor and display are connected to a processing unit or PCB, which is a common practice in electrical engineering and any skilled person will appreciate how this can be implemented.

Software is to be implemented which calculates and reports the likelihood of an ulcer based on the data obtained from the sensor, the method steps to achieve this, are illustrated in the description above, and the implementation thereof may be considered known for a computer scientist. The required calculations are preferably based on results obtained from collecting and analyzing large amounts of (training) data that are collected a priori with the same device. This data is to be labeled by experts on the presence and severity of ulcers, together with additional information relevant to the measurement like skin tone and age of patient. As such, a supervised learning routine is used. Finding the optimal model may be done on dedicated computers.

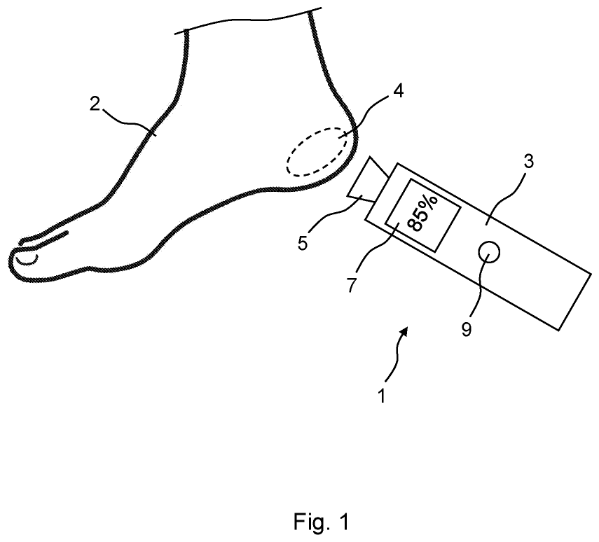

FIG. 1 illustrates a handheld device 1 for identifying pressure ulcers 4 on tissue of a subject for example the foot 2 shown in FIG. 1, with a handheld scanner, the device 1 comprising a radiation source 5 and a radiation detector 5, indication means 7 and a processing unit, (not shown but present within the housing 3 of the device 1). The radiation source and radiation detector are arranged to be placed against the tissue 4 of the subject, for the radiating detector to obtain sample data. The sample data comprises radiation intensity values of radiation emitted by the handheld scanner which reflected of the tissue, and wherein the processing unit is arranged for profiling the sample data, thereby obtaining a reflection spectrum of the sample, and classifying the reflection spectrum using a machine-learning (ML)-classifier thereby obtaining a classification score for identifying the pressure ulcers at the tissue of the subject. Finally, after classifying, the outcome is presented, for example with a display 7 or other indication means. The classification score is thereby presented to a user of the device 1. The ML-classifier is trained with a training set, wherein the training is performed in a training routine; and the training routine comprises training the ML-classifier with a Random Subspace Ensemble routine, and the testing may be initiated with a start button 9.

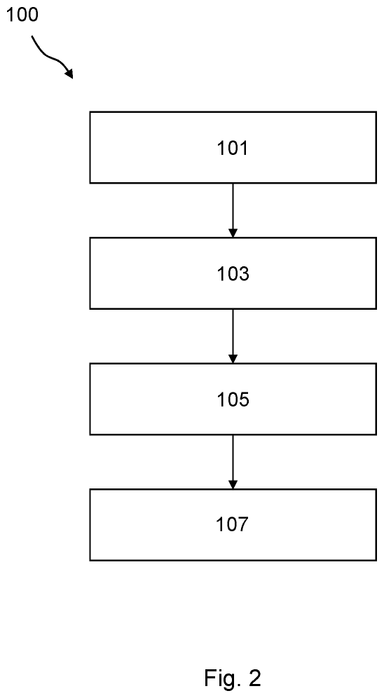

FIG. 2 illustrates a method 100, implemented into a computer, for identifying pressure ulcers or bedsores, decubitus, other on tissue such as a hip, heel or other part of a human body. The method may be performed with a handheld scanner which is preferably operated single handed, the method comprising the steps of:
a) obtaining 101 sample data from the handheld scanner when placed against the tissue of the subject, wherein the sample data comprises radiation intensity values of radiation emitted by the handheld scanner which reflected of the tissue;
b) profiling 103 the sample data, thereby obtaining a reflection spectrum of the sample;
c) classifying 105 the reflection spectrum using a machine-learning (ML)-classifier thereby obtaining a classification score for identifying the pressure ulcers at the tissue of the subject;
d) presenting 107 the classification score by the handheld scanner;
   wherein the ML-classifier is trained with a training set, wherein the
   training is performed in a training routine; and wherein the training routine comprises training the ML-classifier with a Random Subspace Ensemble routine.

To reduce the number of claims, certain aspects of the technology are presented below in certain claim forms, but the applicant contemplates the various aspects of the technology in any number of claim forms. For example, while some aspects of the technology may be recited as a computer-readable medium claim, other aspects may likewise be embodied as a computer-readable medium claim, or in other forms, such as being embodied in a means-plus-function claim.

In the description above, for the purposes of explanation, numerous specific details are set forth in order to provide a thorough understanding of implementations of the disclosed technology. It will be apparent, however, to one skilled in the art that embodiments of the disclosed technology may be practiced without some of these specific details.

Other variations to the disclosed embodiments can be understood and effected by those skilled in the art in practicing the claimed invention, from a study of the drawings, the disclosure and the appended claims. In the claims, the word “comprising” does not exclude other elements or steps, and the indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope thereof.

1. A computer-implemented method for identifying pressure ulcers on tissue of a subject with a handheld scanner, the method comprising the steps of:
a) obtaining sample data from the handheld scanner when placed against the tissue of the subject, wherein the sample data comprises radiation intensity values of radiation emitted by the handheld scanner which reflected off the tissue;
b) profiling the sample data, thereby obtaining a reflection spectrum of the sample data;
c) classifying the reflection spectrum using a machine-learning ulcers (ML)-classifier thereby obtaining a classification score for identifying the pressure in the tissue of the subject; d) presenting the classification score by the handheld scanner; wherein the ML-classifier is trained with a training set, wherein the training is performed in a training routine; and wherein the training routine comprises training the ML-classifier with a Random Subspace Ensemble routine.

2. The computer-implemented method for identifying pressure ulcers on tissue of a subject in accordance with claim 1, wherein the handheld scanner is a Near-InfraRed, NIR, scanner for emitting light in a wavelength range within the near-infrared spectrum.

3. The computer-implemented method for identifying pressure ulcers on tissue of a subject in accordance with claim 1, wherein the handheld scanner is operated in a diffuse reflectance mode to obtain the sample data from the tissue, and which comprises radiation intensity values of radiation emitted by the handheld scanner with an evenly distributed spectral resolution.

4. The computer-implemented method for identifying pressure ulcers on tissue of a subject in accordance with claim 1, wherein the ML-classifier with Random Subspace Ensemble routine is trained in accordance with a duplex method.

5. (canceled)

6. The computer-implemented method for identifying pressure ulcers on tissue of a subject in accordance with claim 1, wherein all the sample data obtained in step a) is assigned to either the training set or a test set, and wherein the training set comprises approximately 70% of the sample data and test data approximately 30% of the sample data.

7. The computer-implemented method for identifying pressure ulcers on tissue of a subject in accordance with claim 1, wherein the training routine comprises training the ML-classifier with a Random Subspace Ensemble routine in which a reflection profile of the tissue is divided into a discrete number of random subspaces.

8. The computer-implemented method for identifying pressure ulcers on tissue of a subject in accordance with claim 7, wherein the discrete number of random subspaces is approximately at least 10.

9. The computer-implemented method for identifying pressure ulcers on tissue of a subject in accordance with claim 6, wherein the ML-classifier comprises a selection of the subspaces which are combined in a combined classifier.

10. The computer-implemented method for identifying pressure ulcers on tissue of a subject in accordance with claim 1, wherein the ML-classifier is trained with a training routine set with an epoch of at least 50.

11. The computer-implemented method for identifying pressure ulcers on tissue of a subject in accordance with claim 1, wherein the ML-classifier comprises approximately at least 10 neurons.

12. A handheld device for identifying pressure ulcers on tissue of a subject with a handheld scanner, the handheld device comprising:
a radiation source,
a radiation detector,
indication means and a processing unit, wherein the radiation source and radiation detector are arranged to be placed against the tissue of the subject, for the radiating detector to obtain sample data, wherein the sample data comprises radiation intensity values of radiation emitted by the handheld scanner which reflected off the tissue, and
wherein the processing unit is arranged for profiling the sample data, thereby obtaining a reflection spectrum of the sample data, and classifying the reflection spectrum using a machine-learning (ML)-classifier thereby obtaining a classification score for identifying the pressure ulcers in the tissue of the subject, and presenting, with the indication means, the classification score to a user of the handheld device, and wherein the ML-classifier is trained with a training set, wherein the training is performed in a training routine; and the training routine comprises training the ML-classifier with a Random Subspace Ensemble routine.

13. The handheld device in accordance with claim 12, wherein the handheld scanner is a Near-InfraRed, NIR, scanner arranged for emitting light in a wavelength range within the near-infrared spectrum.

14. The handheld device in accordance with claim 12, wherein the handheld scanner is arranged to operate in a diffuse reflectance mode to obtain the sample data from the tissue, and which comprises radiation intensity values of radiation emitted by the handheld scanner with an evenly distributed spectral resolution.

15. The handheld device in accordance with claim 12, wherein the ML-classifier is trained in accordance with a Random Subspace Ensemble routine generated in accordance with a duplex method.

16. (canceled)

17. The handheld device in accordance with claim 12, wherein all the sample data obtained by the radiation detector is assigned to either the training set or a test set, and wherein the training set comprises approximately 70% of the sample data and the test data approximately 30% of the sample data.

18. The handheld device in accordance with claim 18, wherein the training routine comprises training the ML-classifier with a Random Subspace Ensemble routine in which a reflection profile of the tissue is divided into a discrete number of random subspaces.

19. The handheld device in accordance with claim 18, wherein the discrete number of random subspaces is approximately at least 10.

20. The handheld device in accordance with claim 12, wherein the ML-classifier comprises a selection of the subspaces which are combined in a combined classifier.

21. The handheld device in accordance with claim 12, wherein the ML-classifier is trained with a training routine set with an epoch of at least 50.

22. The handheld device in accordance with claim 12, wherein the ML-classifier comprises approximately at least 10 neurons.

23. (canceled)

24. (canceled)
